(19) United States
(12) Patent Application Publication
(10) Pub. No.: US 2026/0144742 A1
(43) Pub. Date: May 28, 2026

(54) Composition and method for enhancing the healing of scar tissue

(71) Applicant: Guinevere Isabella Perry, Fort Myers, FL (US)

(72) Inventor: Guinevere Isabella Perry, Fort Myers, FL (US)

(21) Appl. No.: 19/430,206

(22) Filed: Dec. 22, 2025

Related U.S. Application Data
(60) Provisional application No. 63/823,908, filed on Jun. 14, 2025.
(60) Provisional application No. 63/862,017, filed on Aug. 12, 2025.

(30) Foreign Application Priority Data
Sep. 16, 2025 (GB) GB2513702.7

(51) Int. Cl. A61K 9/00; A61K 31/122; A61K 31/355; A61K 31/593; A61K 31/60; A61K 47/10; A61K 47/14; A61P 17/02

(52) U.S. Cl. CPC A61K 9/0014; A61K 9/0053; A61K 31/122; A61K 31/355; A61K 31/593; A61K 31/60; A61K 47/10; A61K 47/14; A61P 17/02

(57) ABSTRACT

A composition for enhancing scar tissue healing and/or reducing pain and inflammation is disclosed. The composition comprises a first active ingredient including salicylic acid in an anti-microbial topical mixture, and a second active ingredient comprising an admixture of at least two vitamins. The composition is formulated such that the combined action of the topical and vitamin components is effective to promote scar tissue healing and mitigate pain and inflammation.

### CROSS-REFERENCE TO RELATED APPLICATIONS

This non-provisional patent includes inventions claimed in U.S. PROVISIONAL APPLICATION 63/823,908 and U.S. PROVISIONAL APPLICATION 63/862,017. Additional this patent claims priority to claims previously filed in the foreign non-provisional UK patent GB2513702.7. This patent contains no research and/or data related to a federal fund or grant.

### TECHNICAL FIELD

The present invention relates to a composition for enhancing scar tissue healing and/or reducing pain and inflammation.

### BACKGROUND

Scar tissue includes forms from fibrous tissue during the body healing process. This process can occur anytime the skin is cut and/or injured after surgical procedures, trauma, or infection. Skin cells use mitosis and the inclusion of collagen to generate new tissue. However, if during this process of healing the skin has excess collagen or the skin forms differently than the surrounding healthy skin the patient will form scar tissue. Once scar tissue has formed this tissue rarely heals on its own without the use of common treatment methods that include corticosteroids, silicone injections, silicone sheets, or surgery.

Scar tissue includes raised scars such as hypertrophic scars and keloids. Both types of scar tissue can be raised above the surrounding skin, but keloids typically are at least 4 mm above the skin and larger in size. Depending on the location the scar can cause pain or limit movement if the keloid is located on joint tissue. Individuals of black, hispanic, or asian ancestry or more likely to develop keloid from tissue injuries or post-surgical procedures. The treatment for the tissue is expensive and invasive and not always effective. The treatment typically reduces the intensity or visibility of the scar tissue but does not heal the tissue.

### SUMMARY

A first aspect provides a composition for enhancing scar tissue healing and/or for reducing pain and inflammation in a subject, the composition comprising a first active ingredient and a second active ingredient, wherein the first active ingredient comprises salicylic acid and the second active ingredient comprises an admixture of at least two vitamins.

An alternative first aspect provides: use of a composition comprising a first active ingredient and a second active ingredient, wherein the first active ingredient comprises salicylic acid and the second active ingredient comprises an admixture of at least two vitamins, in the manufacture of a medicament for enhancing scar tissue healing and/or reducing pain and inflammation in a subject; or a composition comprising a first active ingredient and a second active ingredient, wherein the first active ingredient comprises topical anesthetic and the second active ingredient comprises oral supplement of at least two vitamins, for use in enhancing scar tissue healing and/or reducing pain and inflammation in a subject.

A second aspect provides a method of enhancing scar tissue healing and/or reducing pain and inflammation in a subject, said method comprising the steps of administering to the subject a therapeutically effective amount of a composition comprising a first active ingredient and oral ingestion of a second active ingredient, wherein the first active ingredient comprises salicylic acid and the second active ingredient comprises an admixture of at least two vitamins.

A third aspect provides a method of enhancing scar tissue healing in a subject, said method comprising the steps of administering to the subject a therapeutically effective amount of a composition comprising a first active ingredient and a second active ingredient, wherein the first active ingredient comprises salicylic acid and the second active ingredient comprises an admixture of at least two vitamins.

An alternative third aspect provides: use of a composition comprising a first active ingredient and a second active ingredient, wherein the first active ingredient comprises salicylic acid and the second active ingredient comprises an admixture of at least two vitamins, in the manufacture of a medicament for enhancing scar tissue healing in a subject: or a composition comprising a first active ingredient and a second active ingredient, wherein the first active ingredient comprises salicylic acid and the second active ingredient comprises an admixture of at least two vitamins.

A fourth aspect provides a method for reducing pain and inflammation in a subject due to scarred tissue, said method comprising the steps of administering to the subject a therapeutically effective amount of a composition comprising a first active ingredient and a second active ingredient, wherein the first active ingredient comprises salicylic acid and the second active ingredient comprises an admixture of at least two vitamins.

An alternative fourth aspect provides: use of a composition comprising a first active ingredient and a second active ingredient, wherein the first active ingredient comprises salicylic acid and the second active ingredient comprises an admixture of at least two vitamins, in the manufacture of a medicament for reducing pain and inflammation in a subject; or a composition comprising a first active ingredient and a second active ingredient, wherein the first active ingredient comprises salicylic acid and the second active ingredient comprises an admixture of at least two vitamins for use in reducing pain and inflammation in a subject.

A fifth aspect provides a method of treating scar tissue in a subject, comprising applying to the wound a therapeutically effective amount of a composition comprising a first active ingredient and oral ingestion of a second active ingredient, wherein the first active ingredient comprises salicylic acid and the second active ingredient comprises an admixture of at least two vitamins.

An alternative fifth aspect provides: use of a composition comprising a first active ingredient and a second active ingredient, wherein the first active ingredient comprises wherein the first active ingredient comprises salicylic acid and the second active ingredient comprises an admixture of at least two vitamins, in the manufacture of a medicament for treating scar tissue in a subject; or a composition comprising a first active ingredient and a second active ingredient, wherein the first active ingredient comprises salicylic acid and the second active ingredient comprises an admixture of at least two vitamins, for use in treating scar tissue in a subject.

A sixth aspect provides a kit comprising a composition comprising a first active ingredient and a second active ingredient, wherein the first active ingredient comprises salicylic acid and the second active ingredient comprises an admixture of at least two vitamins.

A seventh aspect provides a kit comprising the first active ingredient and second active ingredient, the first active ingredient comprises salicylic acid and the second active ingredient comprises an admixture of at least two vitamins.

An eighth aspect provides a method of producing a composition for enhancing scar tissue healing and/or for reducing pain and inflammation in a subject, comprising combining a first active ingredient with a second active ingredient, wherein the first active ingredient comprises salicylic acid and the second active ingredient comprises an admixture of at least two vitamins.

In one embodiment, enhancing scar tissue healing comprises enhancing epidermal healing.

### BRIEF DESCRIPTION OF THE DRAWINGS

The invention does not require a drawing or figure to use or understand the invention. The photographs are used to show that the invention was effective and in use by the inventor.

### DETAILED DESCRIPTION

This invention is not limited to specific methods, and variations are possible. The terminology herein describes embodiments and does not restrict the invention beyond the appended claims.

It is to be clearly understood that although prior art publication(s) may be referred to herein, this reference does not constitute an admission that any of these documents forms part of the common general knowledge in the art in the United States or in any other country.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional chemistry and pharmacology within the skill of the art.

In the claims which follow and in the preceding description of the invention, except where the context requires otherwise due to express language or necessary implication, the word “comprise” or variations such as “comprises” or “comprising” is used in an inclusive sense, ie. to specify the presence of the stated features but not to preclude the presence or addition of further features in various embodiments of the invention. It must be noted that as used herein and in the appended claims, the singular forms “a”, “an”, and “the” include plural reference unless the context clearly dictates otherwise. Thus, for example, a reference to “a vitamin” includes a plurality of such vitamins, and a reference to “a herb” is a reference to one or more herb, and so forth. Unless defined otherwise, all technical and scientific terms used herein have the same meanings as commonly understood by one of ordinary skill in the art to which this invention belongs.

In an embodiment, the composition may be used in the treatment of scar tissue. The scar tissue may be any type of scar tissue in any part of the body. As used herein, a “scar tissue” is a region of damaged tissue that can include hypertrophic tissue, hypotrophic tissue, and/or keloid tissue. The damaged tissue may be due to trauma [e.g. mechanical, such as scarred tissue from surgical procedures (including incisions, tooth extractions, or other surgical procedures), scarred tissue from accidents], infection, and/or inflammation. Examples of scar tissue include: scarred tissue resulting from an incision such as cutting instrument (e.g. incision in surgery), lacerations (typically caused by blunt or broken instrument), puncture wounds, abrasions, burn wounds resulting from exposure to heat, electricity, radiation (for example, sunburn and laser surgery); scarred tissue resulting from surgical procedures such as tooth extraction; caustic chemicals; scarred tissue due to aging or the environment, including for example split, dry skin; ulcers (lesion on the surface of the skin or a mucous surface); scarred tissue resulting from wound healing complications associated with subjects suffering from Diabetes Mellitus.

In one embodiment, the scar tissue is a hypertrophic scar or a keloid scar. As used herein, keloids are scar tissue that is raised at least 4 mm from the skin and exceed the original site of trauma. As used herein, hypertrophic scars are scars that are raised above the skin, but can include scars that are not significantly raised above the skin, but are thicker tissues than the surrounding normal skin. Scar tissue may be non-healing areas of the skin, and may occur more in medically compromised patients, individuals of black, hispanic, or asian ancestry.

The inventor has found that the composition described herein, when applied to wounds, results in rapid healing of scar tissue and/or a reduction in the size and thickness of the scar tissue. The inventor has found that the composition described herein enhances healing of underlying tissue including subdermal tissue. Without wishing to be bound by theory, the inventor believes that it is the enhancement, by the composition described herein, of improving liver function and cellular mitosis of underlying tissue which aids in the enhanced healing of the visible scar tissue, and in some embodiments, may result in less scarring in future exposure to skin trauma.

It will be appreciated that the term “enhancing”, “enhance”, and “enhancement” refers to an increase in the rate of healing of the subject as demonstrated by more complete healing at predetermined time from the commencement of treatment of the wound. Similarly, the terms “treating,” “treatment” and the like are used herein to mean affecting the tissue or cells of an individual to increase the rate of healing as demonstrated by more complete healing at predetermined time from the commencement of treatment of the scar tissue.

It will be appreciated that the terms “reducing”, “reduce”, and “reduction” refer to a decrease in the amount of pain felt by a patient as judged by the patient (subject) on a scale of 1 to 10 over the course of treatment and a decrease in inflammation refers to a lessening in the visible signs of inflammation at a predetermined time from the commencement of treatment.

As used herein “anti-microbial mixture” are concentrated agents that contain a composition of salicylic acid formulated to kill microbes and protect the skin. It was observed that use of the salicylic acid compound at the site of scar tissue reduced excess oil production in the area. The skin hardened then began to shrink into the skin. This initiated the healing process and healthy tissue formed under the skin and revisible wound reduction could be seen. Further, while not wishing to be bound by theory, it is believed that formulations in accordance with the invention allow the skin to soften and facilitates rapid transition among macrophages to degrade the scarred tissue and replace it with healthier tissue in which scar tissue healing is enhanced and pain and inflammation are reduced. The ratio or composition of the mixture can range from a 1:1, 2:1, 1:2, or within such parameters as to enhance scar healing. The salicylic acid is an organic compound with a molecular structure of C₇H₆O₃.

The composition comprises a second active ingredient comprising an admixture of vitamins.

The term “vitamin” as used herein refers to an organic substance other than proteins, carbohydrates, and fats that is an essential constituent of the food of the animal. For the most part vitamins are substances that play an essential part in animal metabolic processes but which the animal cannot synthesize. However, certain animals can synthesize certain compounds of this group and all animals needing vitamin D can synthesize it in the presence of UV light. The vitamins are a well characterized group and are generally named using letters of the alphabet. Specific examples are vitamin D3 and K2. In an embodiment, the vitamins in said admixture of vitamins and minerals comprises vitamin D vitamin K.

The composition will be administered as a therapeutically effective amount to a subject for enhancing wound healing. Proportionately smaller or larger doses can be appropriate for subjects having lesser or greater body weight. Such a dose can be administered as needed, but typically administration 1 to 10 times per day, in most cases 1 or 2 times a day, provides adequate continuing relief of pain.

A “therapeutically effective amount” as used herein is intended to mean the amount administered to a subject which when tested in a standardized test involving a female human subject. In one such test as described herein below, the subject rated her pain and the discomfort of the scar tissue on a scale of 1 to 10. The skilled artisan will appreciate, however, that other approaches can be used to assess the severity of pain and relief from such pain.

The terms “subject” or “individual” are used interchangeably herein to refer to any member of the class Mammalia, including, without limitation, humans and other mammals such as primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs. The terms do not denote a particular age. Thus, both adult and newborn individuals are intended to be covered.

Thus provided is the treatment for administration to mammals such as humans, as well as those mammals of economic importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses.

Therefore, one aspect of the present invention involves a method of enhancing wound healing and/or for reducing pain and inflammation in which a composition comprising the composition of the present invention is administered to a subject, in a formulation which provides more complete healing at predetermined time from the commencement of treatment of the wound. The invention is not limited to use of any particular type of formulation. Examples of suitable formulation types are described below.

The terms “administration”, “administering”, and “administered” are used herein interchangeably. Subject to the limitations inherent in treatment of wounds, such as epidermal and mucosal lesions, including trauma or incisions, the composition of the present invention may be administered topically in dosage unit formulations containing conventional non-toxic pharmaceutically acceptable carriers, adjuvants, and vehicles. In some embodiments the composition of the present invention is administered together with a pharmaceutically acceptable carrier or diluent compatible with the composition. In preparing such composition, any conventional pharmaceutically acceptable carrier can be utilized.

The carrier material can be an organic or inorganic inert carrier material suitable for oral administration of the vitamins. Suitable carriers include water, gelatin, gum Arabic, lactose, starch, magnesium stearate, talc, vegetable oils, polyalkylene-glycols, glycerol and the like. Furthermore, the pharmaceutically active preparations may contain other pharmaceutically active agents. Additionally, additives such as flavoring agents, preservatives, stabilizers, emulsifying agents, buffers and the like may be added in accordance with accepted practices of pharmaceutical compounding.

For topical administration to the skin or mucous membrane the composition of the present invention may be prepared as an ointment, tincture, cream, gel, solution including a mouthwash, lotion, spray, aerosol, dry powder for inhalation, suspension, antiseptic and the like.

In fact, any conventional methods of preparing topical compositions can be utilized in this invention.

Among the preferred methods of applying the composition of the present invention is in the form of antiseptic isopropanol solution, mouthwash, ointment, gel, cream, lotion, spray, aerosol, or dry powder, particularly a mouthwash or a gel. A pharmaceutical preparation for topical administration to the skin can be prepared by mixing the composition of the present invention with non-toxic, therapeutically inert, solid or liquid carriers customarily used in such preparation.

In preparing the topical preparations described above, additives such as preservatives, thickeners, perfumes and the like conventional in the art of pharmaceutical compounding of topical preparation can be used. In addition, conventional antioxidants or mixtures of conventional antioxidants can be incorporated into the topical preparations containing the active agent. Among the conventional antioxidants which can be utilized in these preparations are include N-methyl-α-tocopherolamine, tocopherols, butylated hydroxyanisole, butylated hydroxytoluene, ethoxyquin and the like.

Ointment formulations containing the composition of the present invention may comprise admixtures of a semi-solid petroleum hydrocarbon with a solvent dispersion of the composition. Cream compositions containing the composition of this invention may comprise emulsions formed from a water phase of a humectant, a viscosity stabilizer and water, an oil phase of a fatty acid alcohol, a semi-solid petroleum hydrocarbon and an emulsifying agent and a phase containing the composition dispersed in an aqueous stabilizer-buffer solution.

Stabilizers may be added to the topical preparation. Any conventional stabilizer can be utilized in accordance with this invention. In the oil phase, fatty acid alcohol components function as a stabiliser. These fatty acid alcohol components are derived from the reduction of a long-chain saturated fatty acid containing at least 14 carbon atoms.

### EXAMPLES

A 45-year-old subject with suspected Hashimoto's disease had an emergency surgical procedure that left a large scar starting from the pelvic area to the mid torso area. The healed slowly and left a large keloid along several areas of the original incision area. The scarred tissue hurt her periodically and itched constantly. The large keloid caused the subject pain when she wore any clothing that brushed against the keloid.

The subject took Vitamin D3 (25 mcg) and Vitamin K2, additionally the subject wiped the wound twice a day with an antiseptic compound that contained salicylic acid.

### Oral Composition

Vitamin D3 25 mcg
Vitamin K2 100 mg

### Topical Composition

Antiseptic containing isopropanol and salicylic acid.

### IN CONCLUSION

Following treatment with the composition described herein, the patient has remained free from additional scarring. The prior scar tissue continues to heal and improve in texture and elasticity. New literature suggests D3 supplements can inhibit Cutibacterium and/or fungal skin pathogens (see references). A microbial organism found on the skin of almost all humans. There is also emerging literature that suggest Cutibacterium can cause infection, though no current literature have linked them to the formation of hypertrophic scars and/or keloids, some literature suggest they may play a role in the development of hypertrophic scarring commonly associated with acne. This relationship may be related to the bacteria's ability to produce extracellular vesicles (EV) that are absorbed into skin tissue due to the lipid membrane of the EV.

Common literature on the healing of keloid focuses on adding moisture to a wound or scar. Initially the inventor tried hydroscopic compounds as suggested in literature, but it was the combination of both active ingredients in the claim that helped heal the scars. Salicylic acid can kill Cutibacterium and/skin fungus and increase microbial diversity on the skin, see references.

### REFERENCES

1. Alhetheli, G., Elneam, A. I. A., Alsenaid, A., & Al-Dhubaibi, M. (2020). Vitamin D levels in patients with and without acne and its relation to acne severity: a case-control study. Clinical, cosmetic and investigational dermatology, 759-765.
2. Chen, Q., Hou, S., Wu, X. Y., Bu, W. B., Zhou, B. R., & Chen, X. D. (2025). Microbial analyses of infectious keloids on the anterior chest—a case-control study. Archives of Dermatological Research, 317(1), 199.
3. Cheung, C. T., Lancien, U., Corvec, S., Mengeaud, V., Mias, C., Véziers, J., . . . & Dréno, B. (2024). Pro-inflammatory activity of Cutibacterium acnes phylotype IA1 and extracellular vesicles: An in vitro study. Experimental Dermatology, 33(8), e15150.
4. Essam, R., Nasr, M., Khater, M. W., Fayez, B., & Anis, N. (2024). Anti-microbial impact of non-antibiotic agents; salicylic acid, N-acetylcysteine, and isotretinoin against Cutibacterium acnes in patients with acne vulgaris. Archives of Dermatological Research, 317(1), 155.
5. Fang, Q. Q., Wang, X. F., Zhao, W. Y., Chen, C. Y., Zhang, M. X., Shi, B. H., & Tan, W. Q. (2018). The source of ACE during scar formation is from both bone marrow and skin tissue. The FASEB Journal, 32(9), 5199-5208.
6. Felix Montero-Julian, PhD (2021) Cutibacterium Acnes Gram Positive Bacteria Causing Infection, bioMérieux industrial microbiology
7. Gianatasio, C., Abrouk, M., & Waibel, J. S. (2021). Treatment approaches for treating hypertrophic scars and keloids. Dermatological Reviews, 2(1), 11-22.
8. Heppt, M. V., Breuninger, H., Reinholz, M., Feller-Heppt, G., Ruzicka, T., & Gauglitz, G. (2015). Current strategies in the treatment of scars and keloids. Facial Plastic Surgery, 31(04), 386-395.
9. Mustoe, T. A. (2004). Scars and keloids. Bmj, 328(7452), 1329-1330.
10. Perry, Guinevere (2026) Non-Invasive Management of Keloid Scars: Methyl Salicylate Topical Application and Vitamin D3 Supplementation. Journal of Keloid Research. Volume 10
11. Swenson, A., Paulus, J. K., Jung, Y., Weiss, S., Berman, B., Peeva, E., . . . & Jagun, O. (2024). Natural history of keloids: a sociodemographic analysis using structured and unstructured data. Dermatology and Therapy, 14(1), 131-149.
12. Tuffley, M. (2019). U.S. Pat. No. 10,300,028. Washington, DC: U.S. Patent and Trademark Office.
13. van den Broek, L. J., Limandjaja, G. C., Niessen, F. B., & Gibbs, S. (2014). Human hypertrophic and keloid scar models: principles, limitations and future challenges from a tissue engineering perspective. Experimental dermatology, 23(6), 382-386.
14. Yu, T., Chen, J., Wu, S., Jiang, M., Han, L., & Ma, Y. (2024). Potential functionality of Cutibacterium acnes extracellular vesicles in atopic dermatitis and acne vulgaris: A comparative proteomic analysis. PROTEOMICS—Clinical Applications, 18(5), 2300106.

1. A composition for enhancing scar tissue healing, comprising:
(a) a first active ingredient consisting of an anti-microbial topical mixture comprising salicylic acid; and
(b) a second active ingredient consisting of an admixture of at least two vitamins;
wherein the composition includes the first and second active ingredients in amounts effective to enhance scar tissue healing.

2. The composition of claim 1, wherein the anti-microbial topical mixture comprises
one or more anti-microbial compounds selected from the group consisting of methyl salicylate and derivatives thereof.

3. The methyl salicylate of claim 2, wherein the concentration of the topical mixture
is 0.5% to 25%.

4. The composition of claim 1, wherein the anti-microbial topical mixture further
comprises one or more hygroscopic compounds selected from the group consisting of vitamin E and vitamin D.

5. The composition of claim 1, wherein the admixture of at least two vitamins comprises vitamin E and vitamin D.

6. A composition for reducing pain and inflammation, comprising:
(a) a first active ingredient consisting of an anti-microbial topical mixture comprising salicylic acid; and
(b) a second active ingredient consisting of an admixture of at least two vitamins;
wherein the composition includes the first and second active ingredients in amounts effective to enhance scar tissue healing.

7. A method for enhancing scar tissue healing and/or reducing pain and inflammation in a subject, comprising:
(a) topically applying to the skin, at least once daily, a first active ingredient comprising an anti-microbial mixture including salicylic acid, in an amount effective to inhibit the growth of extracellular vesicle (EV)-producing bacteria on
the skin; and
(b) orally administering, once daily, a second active ingredient comprising an
admixture of at least two vitamins, in an amount effective to inhibit the growth of
EV-producing bacteria on the skin; wherein the combined topical and oral administration enhances scar tissue healing and/or reduces pain and inflammation in the subject.
